(19) United States
(12) Patent Application Publication
(10) Pub. No.: US 2005/0003516 A1
(43) Pub. Date: Jan. 6, 2005

(54) Enzymatic treatment of paper making

(75) Inventors: Henrik Lund, Vaerloese (DK); Hanne Hoest Pedersen, Lyngby (DK); Hui Xu, Wake Forest, NC (US); Jing Luo, Raleigh, NC (US)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Novozymes North America, Inc., Franklinton, NC (US)

(21) Appl. No.: 10/817,292

(22) Filed: Apr. 2, 2004

Related U.S. Application Data
(60) Provisional application No. 60/463,279, filed on Apr. 16, 2003.
(60) Provisional application No. 60/516,578, filed on Oct. 31, 2003.

(51) Int. Cl. C12N 9/88

(52) U.S. Cl. 435/278; 162/174

(57) ABSTRACT

The invention relates to processes for treating a pulp, and for making a paper material such as paper, cardboard, linerboard, corrugated paperboard, tissue, towels, corrugated containers, boxes etc., these processes comprising an alkaline treatment of a pulp, a treatment with a pectin lyase and/or a pectate lyase, and, if desired, a draining of the pulp. Pectate lyase in combination with pectinesterase may be substituted for pectin lyase. The invention also relates to the use of xylanase, pectin lyase, pectate lyase, and/or the combination of pectate lyase with pectinesterase for anionic trash reduction and/or reduction of cationic demand of a paper pulp.

### CROSS-REFERENCE TO RELATED APPLICATIONS

This application claims the benefit of U.S. Provisional application No. 60/463,279, filed Apr. 16, 2003, and 60/516,678, filed Oct. 31, 2003 the contents of which are fully incorporated herein by reference.

### FIELD OF THE INVENTION

The present invention relates to processes for making a paper material, for treating pulp, and to pulp washing processes, these processes comprising an alkaline treatment of a pulp, a treatment with a pectin lyase, a pectate lyase, or a pectate lyase in combination with a pectinesterase, and, if desired, a draining of the pulp. The invention also relates to the use of these enzymes, and/or xylanase for anionic trash reduction and/or reduction of cationic demand of a paper pulp.

### BACKGROUND OF THE INVENTION

Paper materials such as paper, cardboard, linerboard, corrugated paperboard, tissue, towels, corrugated containers or boxes, etc. are made from plant fibers. A pulp is an aqueous mixture of such fibers. Pectin, or homogalacturonan, is a constituent of plant fibers, viz. a plant cell wall polysaccharide with a backbone of alpha-1,4-linked galacturonic acid monomers, part of the free carboxylic acid groups of which are methyl esterified.

During the pulping process, in particular as a result of alkaline treatment steps, pectin is released from the fiber structure into the aqueous phase. And there it is perceived as a major contributor to a phenomenon known as anionic trash. Anionic trash forms complexes with certain additives, e.g. cationic retention aids, being used to improve retention of fillers etc. in the paper sheet, and cationic flocculants being used in connection with pulp washing steps. These very large polymer complexes tend to attract water molecules and thereby impair the drainage. Furthermore, the draining screens and filters tend to become blocked. And finally, the anionic trash results in an over-consumption of cationic additives.

The present invention sets out to solve these problems.

### BACKGROUND ART

WO 00/55309 discloses the use of certain pectate lyase enzymes in the treatment of mechanical paper-making pulps or recycled waste paper.

U.S. Pat. No. 5,487,812 (EP 512790) proposes to solve papermaking problems due to the presence of pectin by incorporating the enzyme pectinase in the alkali treated pulp. Pectinase is another name of polygalacturonase (EC 3.2.1.15). It is concluded that if pectin can be degraded to monomers, i.e. galacturonic acid, the cationic demand of the system can be eliminated.

The enzymatic degradation of polygalacturonic acids released from mechanical pulp during peroxide bleaching has been studied and reported by Thornton in Tappi J. 1994, 77(3): 161-167.

Reid et al in Enzyme and Microbial Technology 26 (2000) 115-123 confirms Thornton's finding that pectinase can lower the cationic demand and shows that it applies to pulp bleached at industrial scale.

Xylanases are well known in the paper & pulp industry, i.a. for their use in bleach boosting of pulps, see e.g. EP 386888.

The present inventors surprisingly found that other pectin degrading enzymes, viz. pectin lyase (EC 4.2.2.10) and pectate lyase (EC 4.2.2.2), may be used in the alternative to pectinase, even if galacturonic acid is not resulting from the degradation of pectin catalyzed by these enzymes. Furthermore, surprisingly, and contrary to what is stated in the above EP and US patents, the enzyme treatment can indeed take place before the alkaline treatment step.

Pectin lyase as well as pectate lyase cleave the glycosidic linkages between galacturonic acid monomers in pectin by a trans-elimination reaction and generate unsaturated oligomers with a 4,5 carbon-carbon double bond in the non-reducing end. These degradation products exhibit a distinct UV absorbance at 235 nm. The compound 4-deoxy-L-threohex-4-enopyranosyluronic acid is an example of such degradation products. This is in contrast to polygalacturonase which generates saturated oligomer saccharides such as galacturonic acid as hydrolysis products.

The present inventors surprisingly also found that xylanase may be used to reduce the content of anionic trash in a pulp, if desired in combination with at least one pectin degrading enzyme, e.g. polygalacturonase (EC 3.2.1.15), pectin lyase (EC 4.2.2.10), pectate lyase (EC 4.2.2.2), and/or pectin methyl esterase (EC 3.1.1.11).

### SUMMARY OF THE INVENTION

The present invention relates to a process for the treatment of a paper making pulp, the method comprising an alkaline treatment of the pulp, and a treatment of the pulp with pectin lyase, pectate lyase, or a combination of pectate lyase and pectinesterase.

The pectate lyase treatment may follow or be followed by the alkaline treatment, the pectin lyase treatment is followed by the alkaline treatment, or the treatment with a combination of pectate lyase and pectinesterase may follow or be followed by the alkaline treatment.

These are additional aspects of the invention:

A process for making a paper material, the process comprising an alkaline treatment of a pulp; a treatment with pectin lyase, pectate lyase, or a combination of pectate lyase and pectinesterase; and a draining of the pulp.

A method of reducing the content of anionic trash and/or the cationic demand of a pulp, the method comprising an alkaline treatment, and a treatment of the pulp with i) xylanase; and/or ii) pectin lyase, pectate lyase, or a combination of pectate lyase and pectinesterase. The xylanase treatment follows or is followed by the alkaline treatment, the pectate lyase treatment follows or is followed by the alkaline treatment, the pectin lyase treatment is followed by the alkaline treatment, or the treatment with a combination of pectate lyase and pectinesterase follows or is followed by the alkaline treatment. In a particular embodiment, the method comprises steps i) and ii).

A pulp washing process that comprises the step of treating the pulp with a pectin lyase, a pectate lyase, or a combination of a pectate lyase and a pectinesterase.

The use of a xylanase, a pectate lyase, a pectin lyase, and/or the combination of a pectate lyase and a pectin esterase in a pulp for anionic trash reduction and/or reduction of cationic demand.

### BRIEF DESCRIPTION OF THE FIGURES

FIG. 1 illustrates by use of UV spectrometry that the products resulting from the degradation of pectin with pectate lyase differ from the products resulting from the degradation of pectin with pectinase by a distinct UV absorbance at 235 nm.

### DETAILED DESCRIPTION OF THE INVENTION

Paper and Pulp

A pulp (or a papermaking pulp) is an aqueous mixture of fibers of plant origin. The dry matter content (consistency=Dry Solid, w/w) of the pulp may vary within wide limits, and the pulp may contain various other components as is known in the art of pulp and papermaking.

The pulp can be a fresh, so-called virgin pulp, or it can be derived from a recycled source, or it can be a mixture thereof. The pulp may be a wood pulp, a non-wood pulp, a pulp made from waste paper, or any mixture thereof.

A non-wood pulp may be made, e.g., from bagasse, hemp, bamboo, cotton or kenaf.

A waste paper pulp may be made by repulping waste paper such as newspaper, mixed office waste, computer print-out, white ledger, magazines, milk cartons, paper cups etc. Major grades of recycled fibre furnishes are for instance MOW (mixed office waste), SOW (sorted office waste), ONP (old newsprint), WM (waste magazines) and OCC (old corrugated containers).

A wood pulp may be made from softwood such as pine, redwood, fir, spruce, cedar and hemlock, or from hardwood such as maple, alder, birch, hickory, beech, aspen, acacia and eucalyptus. The wood pulp may be mechanical pulp (such as ground wood pulp, GP, (or GW, or GWP), chemical pulp (such as Kraft pulp or sulphite pulp), semichemical pulp (SCP), thermo-mechanical pulp (TMP), chemithermomechanical pulp (CTMP), or bleached chemithermo-mechanical pulp (BCTMP).

Mechanical pulp is manufactured by grinding and refining methods, wherein the raw material is subjected to periodical pressure impulses. TMP is thermomechanical pulp, GWP is groundwood pulp, PGW, or PGWP, is pressurized groundwood pulp, RMP is refiner mechanical pulp, PRMP is pressurized refiner mechanical pulp and CTMP is chemithermomechanical pulp.

Chemical pulp is manufactured by alkaline cooking whereby most of the lignin and hemicellulose components are removed. In Kraft pulping or sulphate cooking sodium sulphide and/or (preferably and) sodium hydroxide are used as principal cooking chemicals. The Kraft pulp may be a bleached Kraft pulp, which may consist of softwood bleached Kraft (SWBK, also called NBKP (Nadel Holz Bleached Kraft Pulp)), and/or hardwood bleached Kraft (HWBK, also called LBKP (Laub Holz Bleached Kraft Pulp)). Other types of chemical pulps are semichemical pulp (SCP), and bleached chemithermomechanical pulp (BCTMP).

In a particular embodiment, the pulp for use in the process of the invention is a mechanical pulp, such as GWP, SCP, TMP, CTMP, or BCTMP.

In another particular embodiment, the pulp for use in the process of the invention is a waste paper pulp, such as ONP.

As stated above, a papermaking pulp may comprise both recycled paper and virgin pulp. The pulp may have a high (above 18%), medium (7-18%), or low (below 7%) consistency. In particular embodiments, the method and the use of the invention are operated at a high, a medium or a low pulp consistency.

In still another particular embodiment, the pulp to be used in the process of the invention is a suspension of mechanical or chemical pulp or a combination thereof. For example, the pulp to be used in the process of the invention may comprise 0%, 10-20%, 20-30%, 30-40%, 40-50%, 50-60%, 60-70%, 70-80%, 80-90%, or 90-100% of chemical pulp. In a particular embodiment, a chemical pulp forms part of the pulp being used for manufacturing the paper material. In the present context, the expression “forms part of” means that in the pulp to be used in the process of the invention, the percentage of chemical pulp lies within the range of 1-99%. In particular embodiments, the percentage of chemical pulp lies within the range of 2-98%, 3-97%, 4-96%, 5-95%, 6-94%, 7-93%, 8-92%, 9-91%, 10-90%, 15-85%, 20-80%, 25-75%, 30-70%, 40-60%, or 45-55%.

In a still further particular embodiment, the pulp to be used in the process of the invention is a combination of a chemical pulp, such as a Kraft pulp, and a waste paper pulp. The mixed pulp may comprise 50-99%, 60-99%, 70-99%, 80-99%, 85-99%, or 90-99% waste paper pulp. The mixed pulp may comprise 1-50%, 1-40%, 1-30%, 1-25%, 1-20%, 1-15%, or 1-10% chemical pulp, such as Kraft pulp.

The term paper material refers to products, which can be made out of pulp, such as paper, cardboard, linerboard, corrugated paperboard, tissue, towels, corrugated containers or boxes, etc.

The process for preparing a paper material may comprise the additional step of forming the resulting fibers into the desired paper material. The process may also comprise a subsequent drying step.

The effect of the draining or dewatering step is to remove water from the papermaking pulp (increase consistency). The draining step usually takes place in the paper machine, the tissue machine or other forming device. The pulp is usually diluted to a consistency of 0.1-2.0% before the draining. In particular embodiments, the pulp consistency before drainage is 0.1-1.8, 0.1-1.6, 0.1-1.4, 0.1-1.2, 0.1-1.0%. The pulp consistency after drainage is usually 15-45%, or 20-40%, or 25-25%.

Pectin may be released from the pulp to the aqueous phase at various stages of a pulping process, notably at alkaline conditions. Alkaline conditions occur, e.g., in connection with alkaline treatments of the pulp. Examples of alkaline treatments are: Bleaching, in particular peroxide bleaching, such as alkaline hydrogen peroxide bleaching; alkaline re-pulping of waste paper pulp; and alkaline hydrosulphite bleaching or brigthening.

In particular embodiments of the alkaline treatment step of the invention, the pH of the pulp is above 7.0, 7.5, 8.0, 8.5, 9.0, 9.5, 10.0, 10.5, or above 11.0. In other particular embodiments, the pH of the alkaline treatment step is in the range of pH 7.5-11.5, 8.0-11.5, 8.5-11.5, 9.0-11.5, 9.5-11.5 or 10.0-11.5.

The invention relates to a process for the treatment of a paper making pulp, a process for making a paper material, and to a method of reducing the cationic demand and/or the content of anionic trash in a pulp, these processes and methods comprising the following steps: a) alkaline treatment of a pulp, b) treatment of the pulp with various enzymes; and, if desired, a draining of the pulp.

In particular embodiments of these processes and this method, (i) the pectate lyase treatment follows the alkaline treatment step; (ii) the pectate lyase treatment is followed by the alkaline treatment step; (iii) the pectin lyase treatment is followed by the alkaline treatment step; (iv) the treatment with a combination of pectate lyase and pectinesterase is followed by the alkaline treatment step; or (v) the treatment with a combination of pectate lyase and pectinesterase follows the alkaline treatment step.

In particular embodiments of the method, (vi) the xylanase treatment follows the alkaline treatment step; or (vii) the xylanase treatment is followed by the alkaline treatment step.

The term “a” as used in connection with the various enzymes, the paper material, the pulp, the alkaline treatment step, the drainage step etc., means “at least one,” viz. one, two, three or even more of the enzymes in question etc. E.g. more than one pectate lyase may be used in step b), and the overall process for making a paper material may comprise more than one alkaline treatment steps, etc.

The term “follows” and “followed by” means that the two steps in question take place no earlier than simultaneously. For example, in embodiment (i), the pectate lyase treatment occurs no earlier than simultaneously with the alkaline treatment, and in embodiment (iii), the alkaline treatment occurs no earlier than simultaneously with the pectin lyase treatment. There may be additional, unspecified, steps between the enzyme treatment step and the alkaline treatment step.

Accordingly, in particular embodiments of the processes and methods of the invention, the pulp is subjected to:
an alkaline treatment and afterwards a pectate lyase treatment;
an alkaline treatment and concomitantly or at least partly overlapping therewith a pectate lyase treatment;
a pectate lyase treatment and afterwards an alkaline treatment;
a pectin lyase treatment and afterwards an alkaline treatment,
a pectin lyase treatment and concomitantly or at least partly overlapping therewith an alkaline treatment;
a combined treatment with pectate lyase and pectinesterase and afterwards an alkaline treatment;
a combined treatment with pectate lyase and pectinesterase and concomitantly or at least partly overlapping therewith an alkaline treatment;
an alkaline treatment and afterwards a combined treatment with pectate lyase and pectinesterase;.
an alkaline treatment and afterwards a xylanase treatment;
an alkaline treatment and concomitantly or at least partly overlapping therewith a xylanase treatment;
a xylanase treatment and afterwards an alkaline treatment.

A characteristic common feature of the enzymes for use according to the invention, is that unsaturated oligomers with a 4,5 carbon-carbon double bond in the non-reducing end result from the enzyme-aided degradation of pectin. These degradation products exhibit a distinct UV absorbance at 235 nm. This is so for each of the enzymes/enzyme combinations for use in step b) of the processes of the invention.

In particular embodiments, the enzymes for use in step b) of the processes according to the invention can be characterized as follows: The ratio of absorbancy at 235 nm relative to the absorbancy at 350 nm (A₂₃₅/A₃₅₀) is above 30, 35, 40, 45, 50, 55, or above 60, with the following reaction conditions: 1 g/l polygalacturonic acid sodium salt substrate, 40 mg enzyme preparation/I, a treatment time of 60 minutes. The method of Example 1 can conveniently be used for this determination, however the pH and temperature should reflect the characteristics of the enzyme in question. Examples of suitable pH values are 3, 4, 5, 6, 7, 8, 9 or 10, for example pH 7. Examples of suitable reaction temperatures are 30° C., 35° C., 40° C., 45° C., 50° C., 55° C., 60° C., 65° C., or 70° C., for example 55° C.

The enzymes can conveniently be added to any holding tank, e.g. to a pulp storing container (storage chest), storage tower, mixing chest or metering chest.

The treatment with pectin lyase, as well as the combined treatment with pectate lyase and pectinesterase, can be performed before or after the bleaching of pulp, and/or in connection with the pulp bleaching process. The treatment with pectate lyase can be performed before or after the bleaching of the pulp and/or during the bleaching process. When carried out in connection with pulp bleaching, the enzymes may be added together with bleaching chemicals such as hydrogen peroxide. Applying oxygen gas, hydrogen peroxide or ozone or combinations thereof may also carry out the bleaching of pulp. The enzyme preparation may also be added together with these substances.

The enzymes can also be added to the circulated process water (white water) originating from bleaching and process water originating from the mechanical or chemimechanical pulping process.

In the present context, the term “process water” comprises i.a. 1) water added as a raw material to the processes of the invention; 2) intermediate water products resulting from any step of the processes; as well as 3) waste water as an output or by-product of the processes. In a particular embodiment, the process water is, has been, is being, or is intended for being circulated (re-circulated), i.e. re-used in another step of the process. The term “water” in turn means any aqueous medium, solution, suspension, e.g. ordinary tap water, and tap water in admixture with various additives and adjuvants commonly used in these processes. In a particular embodiment the process water has a low content of solid (dry) matter, e.g. below 20%, 18%, 16%, 14%, 12%, 10%, 8%, 7%, 6%, 5%, 4%, 3%, 2%, 1% dry, 0.5%, 0.25%, or below 0.1% dry matter (w/w).

The process, method, and use of the invention may be carried out at conventional conditions in the paper and pulp processing. The process conditions will be a function of the enzyme(s) applied, the reaction time and the conditions given.

The enzyme of the invention should be added in an effective amount. By the term “effective amount” is meant the amount sufficient to achieve the desired and expected effect. In a particular embodiment, the enzymes are dosed in an amount of from about 0.1 mg enzyme protein to about 100.000 mg enzyme protein (of each enzyme) per ton of paper pulp.

In particular embodiments the cationic demand is reduced by at least 2%, 4%, 5%, 8%, 9%, 10%, 12%, 14%, 16%, 18%, 20%, 22%, 24%, 26%, 28%, 30%, 32%, or at least 34%, as compared to a non-enzyme-treated control. The method described in Example 2 is a preferred method for use in such determination.

In further particular embodiments, the amount of the enzymes is in the range of 0.00001-20; or 0.0001-20 mg of enzyme (calculated as pure enzyme protein) per gram (dry weight) of lignocellulosic material, such as 0.0001-10 mg/g, 0.0001-1 mg/g, 0.001-1 mg/g, 0.001-0.1, or 0.01-0.1 mg of enzyme per gram of lignocellulosic material. Again, these amounts refer to the amount of each enzyme.

The enzymatic treatment can be done at conventional consistency, e.g. 0.1-10% dry substance. In particular embodiments, the consistency is within the range of 0.1-45%; 0.1-40%; 0.1-35%; 0.1-30%; 0.1-25%; 0.1-20%; 0.1-15%; 0.1-10%; 0.1-85; 0.1-65; or 0.1-5% dry substance. In other particular embodiments, the consistency is within the range of 0.2-20%, 0.2-18%, 0.2-15%, 0.3-15%, 0.3-12%, 0.3-10%, 0.5-10%, 0.5-8%, or 0.5-5%.

The enzymatic treatment may be carried out at a temperature of from about 10 to about 100° C. Further examples of temperature ranges (all “from about” and “to about”) are the following: 20-100° C., 30-100° C., 35-100° C., 37-100° C., 40-100° C., 50-100° C., 60-100° C., 70-100° C., 10-90° C., 10-80° C., 10-70° C., 10-60° C., and 30-60° C., as well as any combination of the upper and lower values here indicated. A typical temperature is from about 20 to 90° C., or 20 to 95° C., preferably from about 40 to 70° C., or 40 to 75° C.

The enzymatic treatment may be carried out at a pH of from about 2 to about 12. Further examples of pH ranges (all “from about” and “to about”) are the following: 3-12, 4-12, 5-12, 6-12, 7-12, 8-12, 9-12, 2-11, 2-10, 2-9,2-8, 4-10, 5-8 as well as any combination of the upper and lower values here indicated. A typical pH range is from about 2 to 11, preferably within the range from about 4 to 9.5, or 6 to 9.

A suitable duration of the enzymatic treatment may be in the range from a few seconds to several hours, e.g. from about 30 seconds to about 48 hours, or from about 1 minute to about 24 hours, or from about 1 minute to about 18 hours, or from about 1 minute to about 12 hours, or from about 1 minute to 5 hours, or from about 1 minute to about 2 hours, or from about 1 minute to about 1 hour, or from about 1 minute to about 30 minutes. A typical reaction time is from about 10 minutes to 3 hours, 10 minutes to 10 hours, preferably 15 minutes to 1 hour, or 15 minutes to 2 hours.

Various additives can be used in the process, method, or use of the invention. Surfactants and/or dispersants are often present in, and/or added to a papermaking pulp. Thus the processes, methods, and uses of the present invention may be carried out in the presence of an anionic, non-ionic, cationic and/or zwitterionic surfactant and/or dispersant conventionally used in a papermaking pulp. Examples of anionic surfactants are carboxylates, sulphates, sulphonates or phosphates of alkyl, substituted alkyl or aryl. Fatty acids are examples of alkyl-carboxylates. Examples of non-ionic surfactants are polyoxyethylene compounds, such as alcohol ethoxylates, propoxylates or mixed ethoxy-/propoxylates, poly-glycerols and other polyols, as well as certain block-copolymers. Examples of cationic surfactants are water-soluble cationic polymers, such as quartenary ammonium sulphates and certain amines, e.g. epichlorohydrin/dimethylamine polymers (EPI-DMA) and cross-linked solutions thereof, polydiallyl dimethyl ammonium chloride (DADMAC), DADMAC/Acrylamide co-polymers, and ionene polymers, such as those disclosed in U.S. Pat. Nos. 5,681,862; and 5,575,993. Examples of zwitterionic or amphoteric surfactants are betains, glycinates, amino propionates, imino propionates and various imidazolin-derivatives. Also the polymers disclosed in U.S. Pat. No. 5,256,252 may be used.

Enzymes

Xylanases (EC 3.2.1.8), official name Endo-1,4-beta-xylanase, alternative name 1,4-beta-D-xylan xylanohydrolase, catalyse the endohydrolysis of 1,4-beta-D-xylosidic linkages in xylans.

Various pectin degrading enzymes are known:

Polygalacturonase (EC 3.2.1.15) catalyzes the random hydrolysis of 1,4-alpha-D-galactosiduronic linkages in pectate and other galacturonans. Examples of other names are: Pectin depolymerase; pectinase; endopolygalacturonase; endo-polygalacturonase; and endo-galacturonase. The systematic name is poly(1,4-alpha-D-galacturonide)glycanohydrolase.

Pectin lyase (EC 4.2.2.10) catalyzes the eliminative cleavage of (1,4)-alpha-D-galacturonan methyl ester to give oligosaccharides with 4-deoxy-6-O-methyl-alpha-D-galact-4-enuronosyl groups at their non-reducing ends. Examples of other names are: Pectin trans-eliminase; polymethylgalacturonic transeliminase; and pectin methyltranseliminase. The systematic name is (1,4)-6-O-methyl-alpha-D-galacturonan lyase.

Pectate lyase (EC 4.2.2.2) catalyzes the eliminative cleavage of (1,4)-alpha-D-galacturonan to give oligosaccharides with 4-deoxy-alpha-D-galact-4-enuronosyl groups at their non-reducing ends. Examples of other names are: Pectate transeliminase; polygalacturonic transeliminase; and endopectin methyltranseliminase. The systematic name is (1,4)-alpha-D-galacturonan lyase.

Pectinesterase (EC 3.1.1.11) catalyzes the reaction: pectin+n H₂O=n methanol+pectate. Examples of other names are: Pectin demethoxylase; pectin methylesterase; and pectin methyl esterase. The systematic name is pectin pectylhydrolase.

Pectate dissaccharide-lyase (EC 4.2.2.9) catalyzes the eliminative cleavage of 4-(4-deoxy-alpha-D-galact-4-enuronosyl)-D-galacturonate from the reducing end of pectate, i.e. de-esterified pectin. Examples of other names are: Pectate exo-lyase; exopectic acid transeliminase; exopectate lyase; and exopolygalacturonic acid-trans-eliminase. The systematic name: is (1-4)-alpha-D-galacturonan reducing-end-disaccharide-lyase.

The EC numbering is according to the Recommendations of the Nomenclature Committee of the International Union of Biochemistry and Molecular Biology on the Nomenclature and Classification of Enzyme-Catalysed Reactions published in Enzyme Nomenclature 1992 (Academic Press, San Diego, Calif., with Supplement 1 (1993), Supplement 2 (1994), Supplement 3 (1995), Supplement 4 (1997) and Supplement 5 (in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650; respectively).

Enzymes for use in the processes and methods of the invention are: Any pectin lyase capable of degrading methylated homogalacturonan, any pectate lyase capable of degrading non-methylated homogalacturonan, and any pectinesterase capable of demethylating methylated homogalacturonan.

In a particular embodiment, the pectin lyase, pectate lyase and/or pectinesterase, has a pH optimum in the range of 3-11, 4-11, 5-11, 6-11, 7-11, 8-11, 9-11; 3-10, 4-10, 5-10, 6-10, 7-10, 8-10; 3-9,4-9, 5-9,6-9, 7-9; 3-8; 4-8; 5-8; 6-8; 3-7;4-7; or 5-7.

In another particular embodiment, the pectin lyase, pectate lyase and/or pectinesterase, has a temperature optimum in the range of 20-100° C., 30-100° C., 40-100° C., 50-100° C., 60-100° C., 70-100° C., 80-100° C.; 20-90° C., 30-90° C., 40-90° C., 50-90° C., 60-90° C., 70-90° C.; 20-80° C., 30-80° C., 40-80° C., 50-80° C., 60-80° C.; 20-70° C., 30-70° C., 40-70° C., 50-70° C.; 20-60° C., 30-60° C., 40-60° C.; 20-50° C., 30-50° C.; or 20-40° C.

Methods of determining pH optimum and temperature optimum are known in the art. Methylated homogalacturonan and non-methylated homogalacturonan are examples of suitable substrates for use in such methods (for pectin lyase as well as pectinesterase, and for pectate lyase, respectively).

In a particular embodiment the enzyme in question is well-defined, meaning that only one major enzyme component is present. This can be inferred e.g. by fractionation on an appropriate Size-exclusion column. Such well-defined, or purified, or highly purified, enzyme can be obtained as is known in the art and/or described in publications relating to the specific enzyme in question.

For the purposes of the invention, the source of the above enzymes including pectin lyase, pectate lyase and pectinesterase is not critical, e.g. the enzymes may be obtained from a plant, an animal, or a microorganism such as a bacterium or a fungus, e.g. a filamentous fungus or a yeast. The enzymes may e.g. be obtained from these sources by use of recombinant DNA techniques as is known in the art. The enzymes may be natural or wild-type enzymes, or any mutant, variant, or fragment thereof exhibiting the relevant enzyme activity, as well as synthetic enzymes, such as shuffled enzymes, and consensus enzymes. Such genetically engineered enzymes can be prepared as is generally known in the art, e.g. by Site-directed Mutagenesis, by PCR (using a PCR fragment containing the desired mutation as one of the primers in the PCR reactions), or by Random Mutagenesis. The preparation of consensus proteins is described in e.g. EP 897985.

Various xylanases are known, e.g. of fungal or bacterial origin. Bacterial xylanases may derive from strains of Bacillus, for example from a strain of Bacillus halodurans, Bacillus pumilus, Bacillus agaradhaerens, Bacillus circulans, Bacillus polymyxa, Bacillus sp., Bacillus stearothermophilus, or Bacillus subtilis; whereas fungal xylanases, including yeast and filamentous fungal xylanases, may be derived, e.g., from the following fungal genera: Aspergillus, Aureobasidium, Emericella, Fusarium, Gaeumannomyces, Humicola, Lentinula, Magnaporthe, Neocallimastix, Nocardiopsis, Orpinomyces, Paecilomyces, Penicillium, Pichia, Schizophyllum, Talaromyces, Thermomyces, or Trichoderma; e.g. the xylanases described in WO 94/01532, EP 686193, EP 716702, and EP 628080.

A pectin lyase derived from Aspergillus aculeatus is described in WO 94/21786. Various pectate lyases are described in WO 99/27083, WO 99/27084, U.S. Pat. No. 6,280,995, U.S. Pat. No. 6,284,524, and WO 00/55309. Pectinesterases derived from Aspergillus aculeatus and Meripilus giganteus are described in WO 94/25575 and WO 97/31102, respectively. Pectate lyase variants are described in WO 02/06442. A pectate disaccharide-lyase may be derived from a strain of Erwinia (e.g. Swiss-Prot Q05526). A polygalacturonase may e.g. be derived from a strain of Aspergillus (e.g. Swiss-Prot no. P26213).

Other examples of these enzymes can be found at the CAZy(ModO) site: Coutinho, P.M. & Henrissat, B. (1999) Carbohydrate-Active Enzymes server at URL: http://afmb.cnrs-mrs.fr/˜cazy/CAZY/index.html. See also Coutinho, P.M. & Henrissat, B. (1999) Carbohydrate-active enzymes: an integrated database approach. In “Recent Advances in Carbohydrate Bioengineering”, H.J. Gilbert, G. Davies, B. Henrissat and B. Svensson eds., The Royal Society of Chemistry, Cambridge, pp. 3-12; and Coutinho, P.M. & Henrissat, B. (1999) The modular structure of cellulases and other carbohydrate-active enzymes: an integrated database approach. In “Genetics, Biochemistry and Ecology of Cellulose Degradation”., K. Ohmiya, K. Hayashi, K. Sakka, Y. Kobayashi, S. Karita and T. Kimura eds., Uni Publishers Co., Tokyo, pp. 15-23. Pectate lyase and pectin lyase are found under the entry polysaccharide lyases, and pectinesterase under the entry carbohydrate esterases. Pectin lyase is classified in polysaccharide lyase family 1, and pectate lyase in either of polysaccharide lyase families 1, 10, 2, 3, and 9. Pectinesterase is classified in carbohydrate esterase family 8.

In a particular embodiment, the xylanase for use according to the invention is derived from Bacillus. In other particular embodiments, it is derived from Trichoderma, Aspergillus, Humicola, or Thermomyces.

In a particular embodiment, the pectate lyase for use according to the invention is derived from Bacillus. In another particular embodiment the pectin lyase for use according to the invention is derived from Aspergillus. Both embodiments include wild-type enzymes, as well as mutants, variants and fragments thereof which retain the enzymatic activity.

The present invention is further described by the following examples which should not be construed as limiting the scope of the invention.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

Various Embodiments

These are particular embodiments of the invention:

A process for making a paper material, the process comprising the following steps: a) an alkaline treatment of a pulp, b) a treatment of the pulp with a pectate lyase, and c) a draining of the pulp, wherein step b) follows step a).

A process for making a paper material, the process comprising the following steps: a) an alkaline treatment of a pulp, b) a treatment of the pulp with a pectate lyase, and c) a draining of the pulp, wherein step a) follows step b).

A process for making a paper material, the process comprising the following steps: a) an alkaline treatment of a pulp, b) a treatment of the pulp with a pectin lyase, and c) a draining of the pulp, wherein step a) follows step b).

A process for making a paper material, the process comprising the following steps: a) an alkaline treatment of a pulp, b) a combined treatment of the pulp with a pectate lyase and a pectinesterase, and c) a draining of the pulp, wherein step a) follows step b).

A process for making a paper material, the process comprising the following steps: a) an alkaline treatment of a pulp, b) a combined treatment of the pulp with a pectate lyase and a pectinesterase, and c) a draining of the pulp, wherein step b) follows step a).

A method of reducing the content of anionic trash in a pulp, the method comprising the steps of a) an alkaline treatment of the pulp, b) a treatment of the pulp with a pectin lyase a pectate lyase, or a combination of a pectate lyase and a pectinesterase, wherein preferably
(i) the pectate lyase treatment follows the alkaline treatment step;
(ii) the pectate lyase treatment is followed by the alkaline treatment step;
(iii) the pectin lyase treatment is followed by the alkaline treatment step;
(iv) the treatment with a combination of pectate lyase and pectinesterase is followed by the alkaline treatment step; or
(v) the treatment with a combination of pectate lyase and pectinesterase follows the alkaline treatment step.

A method of reducing the cationic demand of a pulp, the method comprising the steps of a) an alkaline treatment of the pulp, b) a treatment of the pulp with a pectin lyase a pectate lyase, or a combination of a pectate lyase and a pectinesterase, wherein preferably
(i) the pectate lyase treatment follows the alkaline treatment step;
(ii) the pectate lyase treatment is followed by the alkaline treatment step;
(iii) the pectin lyase treatment is followed by the alkaline treatment step;
(iv) the treatment with a combination of pectate lyase and pectinesterase is followed by the alkaline treatment step; or
(v) the treatment with a combination of pectate lyase and pectinesterase follows the alkaline treatment step.

Use of a pectate lyase in a pulp for anionic trash reduction and/or reduction of cationic demand.

Use of a pectin lyase before an alkaline treatment of a pulp, for anionic trash reduction and/or reduction of cationic demand.

The combined use of a pectate lyase and a pectinesterase before or after an alkaline treatment of a pulp, for anionic trash reduction and/or reduction of cationic demand.

A pulp washing process comprising the step of treating the pulp with a pectin lyase, a pectate lyase, or a combination of a pectate lyase and a pectinesterase, the process preferably further comprising the additional step of thickening the pulp.

A method of reducing the content of anionic trash in a pulp, the method comprising the steps of a) an alkaline treatment of the pulp, b) a treatment of the pulp with a xylanase, the method preferably further comprising step c) treatment of the pulp with a pectin degrading enzyme.

Use of a xylanase in a pulp for anionic trash reduction and/or reduction of cationic demand, preferably also comprising the use of a pectin degrading enzyme.

### EXAMPLES

### Example 1

### Degradation of Pectin with Pectate Lyase and Pectinases

1 g of polygalacturonic acid sodium salt (Sigma, P3850, minimum purity 85%) was dissolved in 1 L of de-ionized (DI) water. Aliquots of the solution were treated with NOVOZYM™ 51019 pectate lyase, and the pectinase preparations PECTINEX™ ULTRA SP-L, and PECTINEX™ 3X-L (all commercially available from Novozymes A/S, Krogshoejvej 36, DK-2880 Bagsvaerd, Denmark). The treatment took place for 60 min. at pH 7.0 and 55° C. Enzyme dosage was 40 mg/L of the three enzyme preparations, respectively. After the treatment, the solutions were acidified with 8% (w/w) phosphorous acid to pH 2.0. The solutions were diluted 10 times with DI water, and then the UV spectrum was determined by a UV-Vis spectrometer.

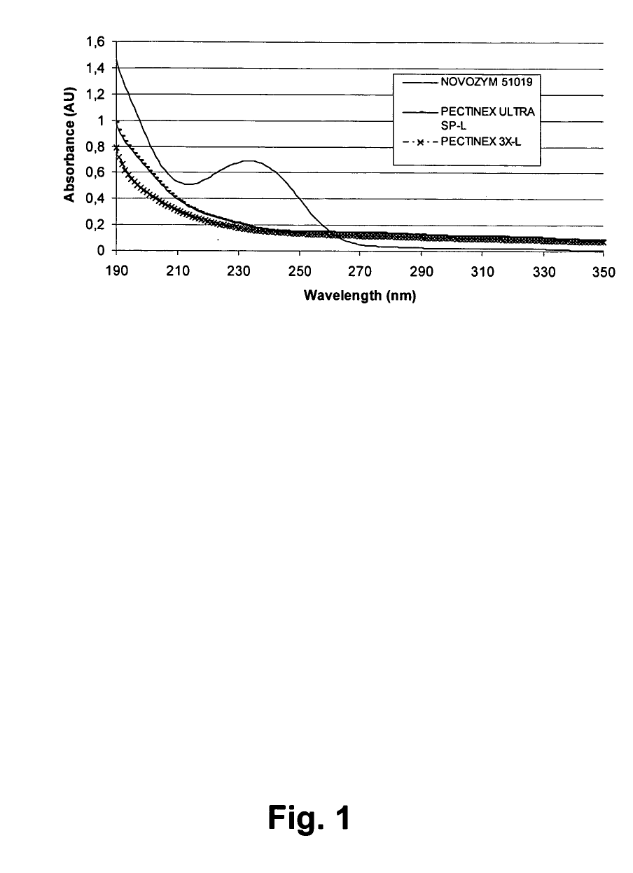

As shown in FIG. 1, the pectate lyase treatment leads to different degradation products as compared to the two pectinases, as evidenced by the characteristic strong UV absorbance at 235 nm. Pectinases degrade pectin into galacturonic acid, whereas pectate lyase degrades demethylated pectin into unsaturated 4-deoxy-L-threo-hex-4-enopyranosyluronic acid group through beta-elimination reactions. The conjugation of the double bond with carboxyl group on C-5 gives rise to the very strong absorption at 235 nm.

### Example 2

### Effect of Pectate Lyase on Cationic Demand after Alkaline Treatment

A thermo-mechanical pulp (TMP) sample was treated with 2% NaOH at 60° C. for 1h. The treated pulp was then filtered through a Brit Jar (200 mesh screen) and the filtrate was neutralized to pH 7 by 0.1 N H₂SO₄. The filtrate was treated with different dosages of the NOVOZYM™ 51019 pectate lyase at 55° C. for 2 hrs.

Cationic demand was determined on all samples using a Mütek particle charge detector and an auto-titrator. 1.0 ml of sample was diluted in 20 ml of DI water and the suspension was titrated with 0.001 N of the cationic retention aid polydiallyldimethyl-ammonium chloride (poly-DADMAC, commercially available from Aldrich).

TABLE 1
Effect of Pectate Lyase on Cationic Demand after Alkaline Treatment

| NOVOZYM ™ 51019 | | | |
|---|---|---|---|
| Pectate Lyase | Cationic Demand, meq/L | STD | % Decrease |
| 0 mg (control) | 0.653 | 0.034 | 0.0 |
| 4 mg/l | 0.541 | 0.031 | 17.2 |
| 20 mg/l | 0.428 | 0.008 | 34.5 |
| 40 mg/l | 0.440 | 0.023 | 32.6 |

### Example 3

### Enzyme Treatment before Alkaline Treatment—Effect on Cationic Demand

A thermo-mechanical pulp (TMP) sample was treated with different dosages of the NOVOZYM™ 51019 pectate lyase, combinations thereof with NOVOSHAPE™ pectinesterase, and with the pectinase preparation PECTINEX™ ULTRA SP-L (all commercially available from Novozymes A/S, Krogshoejvej 36, DK-2880 Bagsvaerd, Denmark). The pulp suspension was adjusted to pH 7.0 before the enzyme treatment. The other enzyme treatment conditions were: 55° C., 4% consistency, for 2 hrs. Then, the pulp samples were further treated with 2% NaOH at 60° C. for 1h. The treated pulp was then filtered through a Brit Jar (200 mesh screen) and the filtrate was neutralized to pH 7 by 0.1 N H₂SO₄.

Cationic demand was determined on all samples using a Mütek particle charge detector and an auto-titrator. 1.0 ml of sample was diluted in 20 ml of DI water and the suspension was titrated with 0.001 N of the cationic retention aid polydiallyldimethyl-ammonium chloride (poly-DADMAC, commercially available from Aldrich).

TABLE 2
Enzyme treatment before alkaline treatment - effect on cationic demand

| | Cationic Demand, | | |
|---|---|---|---|
| Enzymes | meq/L | STD | % Decrease |
| Control | 0.79 | 0.06 | 0.0 |
| NOVOZYM ™ 51019 | 0.69 | 0.04 | 12.7 |
| pectate lyase, | | | |
| 0.5 kg/ton | | | |
| NOVOZYM ™ 51019 | 0.66 | 0.05 | 16.5 |
| pectate lyase, | | | |
| 2.0 kg/ton | | | |
| NOVOZYM ™ 51019 | 0.65 | 0.05 | 17.7 |
| pectate lyase, | | | |
| 0.5 kg/ton, and | | | |
| NOVOSHAPE ™ | | | |
| pectin esterase, 0.5 kg/ton | | | |
| NOVOZYM ™ 51019 | 0.62 | 0.04 | 21.5 |
| pectate lyase, | | | |
| 2.0 kg/ton, and | | | |
| NOVOSHAPE ™ | | | |
| pectinesterase, | | | |
| 2.0 kg/ton | | | |
| PECTINEX ™ Ultra SP L, | 0.76 | 0.03 | 3.8 |
| 0.5 kg/ton | | | |
| PECTINEX ™ Ultra SP L, | 0.72 | 0.02 | 8.9 |
| 2.0 kg/ton | | | |

### Example 4

### Effect of Xylanase on Cationic Demand

The xylanase used in the present example was the PULPZYME HC™ xylanase, commercially available from Novozymes A/S, Krogshoejvej 36, DK-2880 Bagsvaerd, Denmark.

An unbleached CTMP pulp was used.

The pulp was first subjected to an alkaline treatment in the form of a peroxide bleaching at pH starting at 10.5-11.0 and a temperature of 65-85° C. for 60 minutes, using the following chemicals in the amounts indicated: NaOH (100%) 20 lb/ton of pulp; H₂O₂ (100%) 20 lb/ton of pulp; Sodium Silicate solution (technical grade, 40-42° Be, Fisher Scientific) 10 lb/ton of pulp; and DTPA (Diethylenetriaminepentaacetate from Fisher Scientific) 2 lb/ton of pulp.

The alkaline treatment step was followed by a xylanase treatment step (1 kg enzyme per ton of pulp) for 1 hour at 50° C. at a pH of 7. Then the enzyme was deactivated by increasing the temperature to 85° C., holding time 30 minutes.

The thus treated pulp was neutralized to pH 5 by 0.1 N H₂SO₄. A filtrate of the pulp was collected by passing the pulp slurry through a 200 mesh screen supplied and recommended by BTG Mutek. 5.0 ml of filtrate was added to the measuring cell of the detector referred to below, and the suspension was titrated with 0.001 N of the cationic retention aid polydiallyldimethyl-ammonium chloride (poly-DADMAC, commercially available from BTG Mutek).

Cationic demand measurements were measured with a PCD-03 Particle Charge Detector with PCD-2 Titrator manufactured by BTG Mütek as per the operation manual for PCD-03 Particle Charge Detector and PCD-2 Titrator.

A sample treated in the same way except that no xylanase was added was included as a control. The control stood for 1 hour at pH 7 and 50° C. without xylanase.

The results are shown in Table 1 below.

TABLE 1

| Cationic Demand | Control | Xylanase |
|---|---|---|
| [μeq/g] | (No Xylanase treatment) | treatment |
| Test 1 | 57.86 | 50.24 |
| Test 2 | 58.01 | 51.10 |
| Test 3 | 57.73 | 52.45 |
| Test 4 | 57.68 | 52.89 |
| Test 5 | 59.46 | 52.02 |
| Test 6 | 58.32 | 53.77 |
| Average | 58.2 | 52.1 |
| Percentage Reduction | 0 | 10.5% |

### Example 5

### Effect of Xylanase and Pectate Lyase on Cationic Demand

This experiment was conducted as described in Example 4, except that the effect of treatment with pectate lyase was tested in addition to the effect of the xylanase.

The pectate lyase enzyme tested was the NOVOZYM™ 51019 pectate lyase, commercially available from Novozymes A/S, Krogshoejvej 36, DK-2880 Bagsvaerd, Denmark.

The pectate lyase preparation was also dosed at 1 kg/t of pulp.

The results are shown in Table 2.

TABLE 2

| Cationic | Control | | | Pectate Lyase |
|---|---|---|---|---|
| Demand | (No Enzyme | Xylanase | Pectate Lyase | and Xylanase |
| [10⁻⁸ eq/g] | Treatment) | treatment | treatment | treatment |
| Average | 58.2 | 52.1 | 51.1 | 49.8 |
| Percentage | 0 | 10.5% | 12.2% | 14.4% |
| Reduction | | | | |

Another pectate lyase (in what follows designated “Pectate Lyase II”), viz. a variant of the Bacillus subtilis pectate lyase (WO02/092741), showed improved performance, viz. a reduction in cationic demand of 15.0%. The Pectate Lyase II variant is described in Table 6 of WO03/095638, listing the following variants:
D48P+M64F+T105P+K1391+Q146H+K213T+K218P+T2581+A305P+S331P;
K1391+Q146H+S337C;
D48P+M64F+T105P+K1391+Q146H+K213T+K218P+T2581+A305P+S331P+S340P;
D48P+M64F+T105P+K1391+Q146H+K213T+K218P+T2581+A305P+S331P+K334E+S337K⁺ S34 OP;
M64F+K1391+Q146H+S337C;
D48P+M64F+T105P+K1391+Q146H+N189D+K213T+K218P+T2581+S298N+A305P+S331P+S3 37R;
D48P+M64F+T105P+K1391+Q146H+K213T+K218P+T2581+A305P+S331P+S337K;
D48P+M64F+T105P+K1391+Q 146H+K213T+K218P+T2581+A305P+S331P+S337R;
D48P+M64F+T105P+K1391+Q 146H+K148E+K213T+K218P+T2581+A305P+S331P+S337R; and
D48P+M64F+T105P+K1391+Q146H+K213T+K218P+T2581+A305P+S331P+S337K+S340P.

### Example 6

### Effect of Xylanase and Pectate Lyase on Cationic Demand at Increased Temperature

This experiment was conducted as described in Examples 4 and 5, except that the enzyme treatment steps were conducted at 70° C. instead of 50° C. The enzymes were applied in varying dosages (see below). The results are shown in Table 3.

TABLE 3

| | Percentage Reduction of Cationic Demand | | | |
|---|---|---|---|---|
| Enzyme | Control | | Pectate | Pectate |
| Dosage | (No Enzyme) | Xylanase | lyase I | lyase II |
| 0.1 kg/t | 0 | 12.9% | 9.6% | 15.5% |
| 0.05 kg/t | 0 | 9.1% | 7.6% | 13.7% |
| 0.01 kg/t | 0 | 4.1% | 3.2% | 7.8% |

1. A process for the treatment of a paper making pulp, the process comprising the following steps: a) an alkaline treatment of the pulp, b) a treatment of the pulp with a pectin lyase, a pectate lyase, or a combination of a pectate lyase and a pectinesterase.

2. The process of claim 1, wherein
(i) the pectate lyase treatment follows the alkaline treatment step;
(ii) the pectate lyase treatment is followed by the alkaline treatment step;
(iii) the pectin lyase treatment is followed by the alkaline treatment step;
(iv) the treatment with a combination of pectate lyase and pectinesterase is followed by the alkaline treatment step; or
(v) the treatment with a combination of pectate lyase and pectinesterase follows the alkaline treatment step.

3. The process of claim 1, further comprising step c) a draining of the pulp.

4. The process of claim 3 which is a process for making a paper material.

5. The process of claim 1, wherein the enzyme treatment of step b) leads to the formation of unsaturated oligomers with a 4,5 carbon-carbon double bond in the non-reducing end, resulting in degradation products exhibiting a distinct UV absorbance at 235 nm.

6. The process of claim 3, wherein step c) follows steps a) and b).

7. The process of claim 1, which comprises at least one of the following additional steps: d) debarking, e) chipping, f) refining, g) screening, h) cleaning, i) thickening, j) storage, k) forming the paper material, and/or 1) drying the paper material.

8. The process of claim 1, wherein the alkaline treatment is a hydrogen peroxide or hydrosulphite bleaching, or a repulping of recycled pulp.

9. The process of claim 1, wherein the pulp is additionally treated with a polygalacturonase and/or a pectate disaccharide-lyase.

10. The process of claim 1, wherein the enzymes are added to wash water, white water, process water, and/or drained water.

11. The process of claim 1, wherein the enzymes are added together with complexing agents and/or surfactants.

12. A method of reducing the cationic demand and/or the content of anionic trash in a pulp, the method comprising the steps of a) an alkaline treatment of the pulp, b) a treatment of the pulp with i) a xylanase, and/or ii) a pectin lyase, a pectate lyase, or a combination of a pectate lyase and a pectinesterase.

13. The method of claim 12, wherein
(i) the pectate lyase treatment follows the alkaline treatment step;
(ii) the pectate lyase treatment is followed by the alkaline treatment step;
(iii) the pectin lyase treatment is followed by the alkaline treatment step;
(iv) the treatment with a combination of pectate lyase and pectinesterase is followed by the alkaline treatment step;
(v) the treatment with a combination of pectate lyase and pectinesterase follows the alkaline treatment step;
(vi) the xylanase treatment follows the alkaline treatment step; and/or
(vii) the xylanase treatment is followed by the alkaline treatment step.

14. The method of any one of claims 1243, wherein step b) includes a treatment of the pulp with a pectinase.

15. (Cancelled)

16. (Cancelled)
